(19) United States
(12) Patent Application Publication
(10) Pub. No.: US 2003/0130644 A1
(43) Pub. Date: Jul. 10, 2003

(54) Extendable hook fastening system

(76) Inventor: Andrew Thomas Baker, Lawrenceville, GA (US)

Correspondence Address: Christopher C. Campbell, Esq., HUNTON & WILLIAMS, 1900 K. Street, N.W., Washington, DC, 20006-1109, US

(21) Appl. No.: 10/041,229

(22) Filed: Jan. 7, 2002

(51) Int. Cl. A61F 13/15

(52) U.S. Cl. 604/389; 604/391

(57) ABSTRACT

An absorbent garment having a fastener assembly with enhanced flexibility is obtained by providing fastener assemblies with at least one, and preferably a plurality of slits. The slits may have linear portions, curved portion or combination thereof. The degree of flexibility may be varied by the number, type, position and orientation of the slits.

### BACKGROUND OF THE INVENTION

1. Field of the Invention

The present invention relates generally to absorbent garments. In particular the invention relates to an absorbent garment having fasteners that bend in two planes. The fasteners have improved flexibility that facilitates use of the fasteners and provides for improved fit of the garment.

2. Description of Related Art

Absorbent garments, such as diapers and adult incontinence products, often are constructed to have an hourglass or rectangular shape that is fitted between a wearer's legs, wrapped around the wearer's waist, and then sealed on either side to form a pant-like structure on the wearer. To improve fit, many of these garments have an elasticized waist which the caregiver stretches out while placing the garment on the wearer. Fasteners typically are located in the waist region, or on tabs extending therefrom, for attaching one half of the garment to the other half.

Many conventional absorbent articles and diapers in particular, are typically fastened about the waist of the wearer using fasteners that can be releasably engaged together. The fasteners typically include a pair of first fasteners that are located on tabs on the side edges of the rear portion of the absorbent article and at least one second fastener that is located on the front portion of the absorbent garment. In use, the first fasteners are configured to be releasably engageable with the second fastener to fasten the absorbent article about the waist of the wearer.

The process used to fasten such conventional absorbent garments about a wearer typically includes the following steps: (1) placing the absorbent garment in a laid out flat configuration; (2) positioning the back of the torso of the wearer on the rear portion of the absorbent garment; (3) bringing the front portion of the absorbent garment between the legs of the wearer; (4) extending the fasteners attached to the waist portion about the side regions of the waist of the wearer; and (5) releasably engaging the fasteners together to secure the absorbent garment about the waist of the wearer. Such an involved process can be difficult for a care-giver to accomplish.

In order to secure the absorbent garment about wearer's waist, the caregiver typically must grasp the lateral edge of the garment and pull outwards. Often, the only convenient places at which the caregiver can grasp the garment are the laterally distal edges of the fasteners. In doing so, the caregiver places the fasteners under a tensile load that may deform the tabs and the fasteners, thereby inhibiting the proper attachment of the fasteners. In addition, the caregiver may have to pinch the garment between his or her fingers, and the caregiver's fingers or thumbs may partially or completely cover the fasteners while the caregiver attempts to secure the fasteners. As the caregiver affixes some portions of the fasteners, other portions of the fasteners may form wrinkles that are not fully attached to the garment. Other portions of the fastener may be attached while the waist or tab elastic is in different states of stretch, thereby causing different loading in different parts of the fastener. Still other portions of the fastener may not be attached at all, and may even turn to face outward due to the tensile load in the elastics or other loads generated during use. In any of these cases, and others not mentioned herein, the fasteners may not attach the two halves of the garment to one another in an ideal manner, and may slip, loosen, or partially or completely open during use.

Fastening systems heretofore in use have included adhesive fastening, such as shown in U.S. Pat. Nos. 3,630,201; 4,047,530; 4,049,001 and 4,050,453. The fastening device generally comprises an adhesive tab attached to the outer (backing) sheet of the diaper at the rear portion and the active adhesive surface of the tab is stuck onto another portion of the backing sheet surface near the front portion thereby closing the diaper. Once positioned it may be inconvenient if not difficult to release the adhesive tab and reposition it should the initial position be unsatisfactory due to some of the factors noted above.

Mechanical closure systems for absorbent garments have been devised using hook (e.g. “mushroom”) and loop (for example, Velcro.RTM.) fasteners for closure of the waistband, such as is set forth by French Patent No. 2,594,650 and EP 0 276 970 A2. Mechanical closure systems such as hook and loop systems have the advantage of being amenable to multiple fastening and unfastening events without appreciably changing their ability to affect a secure closure. However to have suitable peel, shear and latching characteristics, the hook and loop materials, particularly the hook materials typically are rigid. The rigid hook material may be uncomfortable to the wearer; may not attach with the desired efficiency to the loop material when the loop material is on a surface with a rounding contour such as the tummy of a wearer, e.g. portions of the hooks may fail to attach or attach at an orientation that is less than optimal; and the hook material may lack a “hand” or flexibility desirable for manipulation by the care giver in effecting an opening or closure of the fastening system.

The foregoing description of the various products, methods, and apparatus and their attendant disadvantages is in no way intended to limit the scope of the present invention, or to imply that the present invention does not include some or all of the various elements of the products, methods and apparatus in one form or another. Indeed, various embodiments of the invention may be capable of overcoming some of the disadvantages, while still retaining some or all of the various elements of the products, methods, and apparatus in one form or another.

### SUMMARY OF INVENTION

It would be desirable to provide an absorbent garment with flexible hook fastening tabs. Flexible hook fastening tabs may provide the advantages of multiple attachments of the hook fastening tabs without degradation of fastening ability, while reducing the disadvantages of stiffness and discomfort to the wearer typically associated with hook and loop fastener systems. Further, it would be desirable to provide a hook fastening tab with enhanced flexibility to facilitate caregiver handling and positioning of the tabs in effecting an opening or closure of the fastening system when securing the absorbent garment on the wearer or removing the absorbent garment.

These and other features of an embodiment of the invention are achieved by providing an absorbent garment having a longitudinal dimension and a lateral dimension, the garment comprising: a top sheet and a back sheet, whereby the top sheet and the back sheet form a first waist region, a second waist region longitudinally opposite the first waist region and a crotch region there between. The absorbent garment further comprises an absorbent core disposed between the top sheet and the back sheet; a pair of tabs each extending laterally away from opposite lateral sides of the first waist region. At least one fastening element is attached to each tab in an attachment region and at least one slit is disposed in the attachment region. One or more target devices are disposed to the garment to the garment in the second waist region. The target devices and fastening elements are capable of attaching to one another and the target devices are located so that the first waist and second waist region of the garment may be joined to one another to secure the garment on the intended wearer.

In preferred embodiments, a plurality of slits is disposed in the attachment region. The slits may be disposed in a direction parallel to the lateral dimension, in a direction parallel to the longitudinal direction or in a position non-parallel to either of the longitudinal and lateral dimensions. In preferred embodiments, the fastening element comprises the hook portion of hook and loop fastener. In other embodiments, the fastening element may be an adhesive tape. The one or more slits disposed in the attachment region may penetrate the fastening element or alternatively penetrate both the fastening element and the tab in the region of attachment.

Another feature of an embodiment of the invention is a method of making an absorbent garment having a longitudinal dimension and a lateral dimension, the method comprising: providing a top sheet; providing a back sheet material to overlay the top sheet and thereby form a first waist region, a second waist region longitudinally opposite the first waist region and a crotch region there between. The method further comprises disposing an absorbent core between the top sheet and the back sheet providing a pair of tabs and attaching each tab to the front waist region so that each of the tabs extends laterally away from an opposite lateral side of the first waist region. At least one fastening element is attached to each tab in an attachment region and at least one slit is disposed in the attachment region. One or more target devices are disposed on the second waist region of the garment. The at least one fastening element and the one or more target devices are capable of attaching to one another. The target devices are located so that the first and second waist regions may be joined to one another to secure the garment on an intended wearer. In preferred embodiments a plurality of slits are disposed in the attachment region.

Another feature of an embodiment of the invention includes a fastening system for an absorbent garment. The fastening system for the absorbent garment comprises at least one tab, at least one fastening element attached to each tab in a region of attachment wherein at least one slit is disposed in the attachment region, and one or more target devices wherein the at least one fastening element and the one or more target devices are capable of attaching to one another. In preferred embodiments a plurality of slits are disposed in the attachment region.

A further feature of an embodiment of the invention includes a method of making a fastening system. The method of making a fastening system comprises forming at least one tab; attaching at least one fastening element to each tab is an attachment region, cutting at least one slit in the attachment region and providing one or more target devices, wherein the at least one fastening element and the one or more target devices are capable of attaching to one another.

### BRIEF DESCRIPTION OF THE DRAWINGS

The invention will be described with reference to the attached drawings illustrating exemplary embodiments of the invention. For clarity, features that appear in more than one Figure have the same reference number in each Figure.

FIG. 1 depicts a partially cut away view of an exemplary embodiment of an absorbent garment;

FIG. 2 depicts a typical tab with attached fastening element;

FIG. 3 shows a tab and attached fastening element with slits parallel to the lateral axis according to an exemplary embodiment of the invention;

FIG. 4 depicts a tab and attached fastening element with slits parallel to the longitudinal axis according to an exemplary embodiment of the invention;

FIG. 5, shows a tab and attached fastening element with slits parallel to the longitudinal axis according to an exemplary embodiment of the invention;

FIG. 6 depicts a tab and attached fastening element with curved slits according to an exemplary embodiment of the invention; and

FIG. 7 depicts a tab and attached fastening element having a geometric pattern of slits according to one embodiment of the invention.

### DETAILED DESCRIPTION OF PREFERRED EMBODIMENTS

One advantage of an embodiment of the invention is that by providing a fastening element for an absorbent garment with slits that penetrate the fastening element or penetrate the fastening element and the tab material underlying the fastening element, the fastening element has enhanced flexibility including the ability to bend in two planes. This enhanced flexibility provides an absorbent garment that is more comfortable to the wearer, has improved efficiency of closure for fastening systems such as hook and loop fastening systems and has improved fit. Further enhanced flexibility of fastening elements facilitates manipulation of the fastener system by a caregiver when applying or removing the absorbent garment from a user.

Another advantage of an embodiment of the invention is that by selection of the orientation, type and number of slits provided in the fastener element, the amount of flexibility and orientation of the flexibility can be selected.

A further advantage of some embodiments of the invention is that fasteners with slits may have elastic properties.

As used herein, the term “absorbent garment” refers to garments that absorb and contain exudates, and more specifically, refers to garments that are placed against or in proximity to the body of the wearer to absorb and contain the various exudates discharged from the body. A nonexhaustive list of examples of absorbent garments includes diapers, diaper covers, disposable diapers, training pants, feminine hygiene products and adult incontinence products. The term “disposable absorbent garment” refers to absorbent garments that are intended to be discarded or partially discarded after a single use (i.e., they are not intended to be laundered or otherwise restored or reused). The term “unitary disposable absorbent garment” refers to a disposable absorbent garment that is essentially a single structure (i.e., it does not require separate manipulative parts such as a diaper cover and insert). As used herein, the term “diaper” refers to an absorbent garment generally worn by infants and incontinent persons about the lower torso.

The claims are intended to cover all of the foregoing classes of absorbent garments, without limitation, whether disposable, unitary or otherwise. These classifications are used interchangeably throughout the specification, but are not intended to limit the claimed invention. The invention will be understood to encompass, without limitation, all classes of absorbent garments, including those described herein.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Absorbent garments and diapers may have a number of different constructions. In each of these constructions it is generally the case that an absorbent core is disposed between a liquid pervious body-facing top sheet, and a liquid impervious, exterior facing back sheet. In some cases, one or both the top sheet and the back sheet may be shaped to form a pant-like garment. In other cases, the top sheet, back sheet and absorbent core may be formed using a discreet assembly that is placed on a main chassis and the chassis is made to form a pant-like garment. In the case of diapers, a caregiver usually wraps the diaper around the wearer's waist and joins the side seams manually by attaching one or more adhesive or mechanical tabs, thereby making the pant-like structure. Typically, the tabs are a pair of tabs that extend laterally away from opposite sides of the first waist region. The pair of tabs may be extensions of one or both of the top sheet and back sheet, or may be separate pieces of material. A fastener (also referred to herein as fastener element) is attached to each tab, and one or more target devices are attached to the garment in the second waist region. The fasteners may be attached to the target devices so that the first waist region and second waist region of the garment are joined to one another to secure the garment on a wearer. In one preferred embodiment of the invention, the fastener may be a hook portion of a hook and loop fastener, and the target area may be the loop portion of a hook and loop fastener or the target area may be the outer cover of the absorbent garment. In another embodiment, the fastener may be an adhesive tape and the target devices may comprise a tape receiving surface.

For clarity, the present invention is described herein, with reference to the diaper type garment. The invention may be used with other constructions including for example, the training pant-type garments, adult and incontinent products or feminine hygiene products.

More particularly, the present invention relates to an absorbent garment having at least one and preferably a plurality of slits in the fastener element. These slits may penetrate the fastener element or the fastener element and the underlying tab material. The slits may be linear, curved, or a combination thereof and may be disposed parallel to the longitudinal dimension of the garment, parallel to the lateral dimension of the garment, non-parallel to both the longitudinal and lateral dimension or, for a plurality of slits, a combination thereof.

In preferred embodiments the fastener element is the hook portion of a hook and loop fastener. In another exemplary embodiment the fastener is an adhesive fastener.

The invention also relates to a method of making an absorbent garment having at least one and preferably a plurality of slits in the fastening element. The method comprises providing a top sheet and a back sheet to overlay the top sheet and thereby form a first and second waist region with a crotch region between; disposing an absorbent core between the top sheet and the back sheet; providing a pair of tabs, each extending laterally away from an opposite lateral side of the first waist region, attaching at least one fastening element to each tab in an attachment region and disposing one or more target devices in the second waist region. The fastening element has at least one and preferably a plurality of slits that penetrate the fastening element or the fastening element and the underlying tab material. The slits may be of the type and orientation described above.

The invention relates further to a fastening system for an absorbent garment comprising at least one tab having a fastening element attached to each tab with at least one slit and preferably a plurality of slits disposed in the fastening element; and one or more target devices capable of attachment with the fastener. The slits may be linear, curved, or combinations thereof and may be disposed parallel to one of the tab sides or non-parallel to the tab side or, for a plurality of slits and a combination thereof. The slit may penetrate the fastening element or both the fastening element and the underlying tab material.

The invention also relates to a method of making the fastening system with one or more slits in the fastening element as described herein.

The invention now will be described with reference to the attached drawings illustrating preferred embodiments of the invention. For clarity, features that appear in more than one Figure have the same reference number in each Figure.

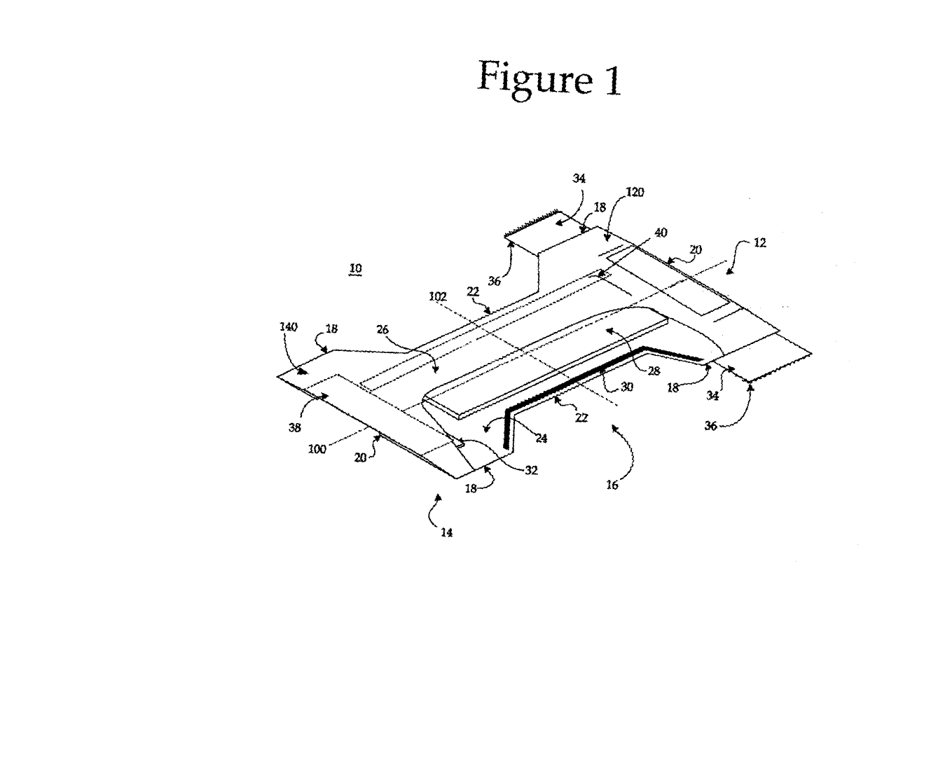

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art will appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their extended condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 forms the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent core 28 preferably is disposed between at least a portion of the top sheet 24 the back sheet 26.

An embodiment of the present invention may further comprise various additional features. One or more pairs of elastic gathers 30 may extend adjacent the crotch edges 22 or a portion thereof. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 also may be equipped with strips of elastic waist foam 32 or other elastically extensible material, which help contract the garment around the wearer's waist, providing improved fit and leakage prevention.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. Due to the wide variety of backing and liner sheet construction and materials currently available, the invention is not intended to be limited to any specific materials or constructions of these components. The top sheet 24 and back sheet can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 may be air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12,14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent core 28. Examples of suitable liner sheet materials include non-woven spunbond or carded webs of polypropylene, polyethylene, nylon, polyester and blends of these materials.

The back sheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent core 28 while outboard non-woven breathable side back sheet panels are attached to the side edges of the central poly back sheet panel. Alternatively, the back sheet 26 may be formed from microporous poly coverstock for added breathability.

The top sheet 24 may be formed of three separate portions or panels. Those skilled in the art will recognize, however, that top sheet 24 need not be made of three separate panels, and that it may be comprised of one unitary item. A first top sheet panel (not shown) may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 14 to the first waist region 12, or a portion thereof.

A second and third top sheet panels (e.g., outer top sheet panels, not shown), in this alternative embodiment may be positioned laterally outside of the central top sheet panel. The outer edges of the outer top sheet panels may substantially follow the corresponding outer perimeter of the back sheet 26. In some embodiments the outer top sheet panels may be substantially liquid-impervious and hydrophobic, in areas away from the crotch area. The material for the outer top sheet portions or panels is preferably polypropylene and can be woven, non-woven, spunbonded, carded or the like, depending on the application.

At the point of connection between the outer top sheet panels and the central top sheet panel, inner edges of the outer top sheet portions or panels may extend upwardly to form waste containment flaps 40. The waste containment flaps 40 preferably are formed of the same material as the outer top sheet portions or panels, as in the embodiment shown. The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner.

The waste containment flaps 40 preferably include a portion that folds over onto itself to form a small enclosure. At least one, and depending on the size of the enclosure sometimes more than one, elastic member may be secured in the enclosure in a stretched condition. When the flap elastic attempts to assume the relaxed, unstretched condition, the waste containment flaps 40 rise above the surface of the central top sheet portion or panel.

The top sheet 24 may be made of any suitable relatively liquid-pervious material currently known in the art or later discovered that permits passage of a liquid there through. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 28. The top sheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make the outer top sheet panels preferably are substantially fluid impervious and hydrophobic, while the remainder of the top sheet 24 (e.g., central top sheet panel) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent core 28, and the back sheet 26 is large enough to prevent leakage from the garment 10. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the top sheet 24 to the back sheet 26. The particular joining method may be dictated by the types of materials selected for the top sheet 24 and back sheet 26.

As mentioned above, the absorbent garment preferably is provided with leg elastics 30 extending through crotch region 16 or a portion thereof, adjacent crotch edge 22. The absorbent garment of the invention also preferably is provided with waist elastic material 32 optionally in the first and or second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1-½ inches in the dimension that parallels the longitudinal axis 100 and about 3-6 inches in the dimension that parallels the lateral axis 102. The foam strips preferably are positioned between the top sheet 24 and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably at least a portion of the foam strips 32 are stretched 50-150%, preferably 100% more than their unstretched dimension before being adhesively secured between the back sheet 26 and top sheet 24.

The underlying structure beneath the top sheet 24 may include, depending on the diaper construction, various combinations of elements, but in each embodiment, it is contemplated that the absorbent garment will preferably include an absorbent core 28. For example, additional layers may be disposed between the top sheet 24 and absorbent core 28, and/or other additional layers may be disposed between these layers, or between absorbent core 28 and back sheet 26. The additional layer(s) may include a fluid transfer layer, a fluid handling layer, a storage layer, a wicking layer, a fluid distribution layer, and any other layer(s) known to those having ordinary skill in the art.

Although the absorbent core 28 depicted in FIG. 1 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the top sheet 24, back sheet 26, or any other suitable part of the garment 10 by any method known in the art, in order to fix the absorbent core 28 in place. In addition to the respective layers in the absorbent core 28, as will be described in greater detail hereinafter, the overall absorbent core 28 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 28 of the invention, using the guidelines provided herein.

Any suitable absorbent material may be used for absorbent core 28. Absorbent cores containing a mixture of fibrous material and superabsorbent polymers (SAP) are well known in the art and described, for example, in U.S. Pat. Nos. 5,281,207, and 6,068,620 to Chmielewski, and U.S. Pat. No. 5,863,288, to Baker, the disclosures of each of which are herein incorporated by reference in their entirety and in a manner consistent with this disclosure. The fibrous material can be any fibrous material capable of absorbing fluids, and capable of retaining SAP particles within its matrix. Preferred fibrous materials may be selected from tow fibers, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. In addition, rayon, Courtauld's LYOCELL, polyacrylonitrile, cotton fibers and cotton linters are alternatively preferred. The remaining fibers, surface-modified polyolefin/polyester bicomponent fibers, and surface-modified polyester/polyester bicomponent fibers are also believed to be effective fibrous materials for use in the invention.

Any superabsorbent polymer (SAP) now known or later discovered may be used in absorbent core 28, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The absorbent garment 10 also includes fastening elements to enable attachment of the first waist region 12 to second waist region 14. The first and second waist regions 12, 14 may be joined to one another by a pair of fasteners 36, or a plurality of fastening elements 36, each of which is attached to a tab 34 protruding from an opposite lateral side edge 18 of the first waist region 12. The fasteners 36 may interact with one or more target devices 38 located in the second waist region 14 to provide sufficient gripping force to hold the garment 10 on a wearer. While the embodiment depicted in FIG. 1 shows the tabs 34 and fasteners 36 being located in the first waist region 12, and the target device 38 being in the second waist region 14, skilled artisans will recognize that these locations may be reversed. Exemplary embodiments of the tabs 34, fasteners 36 and target devices 38 are provided herein, however, the present invention is not intended to be limited to any particular construction or materials for these parts.

The tabs 34, fasteners 36, and target devices 38, preferably are located and sized so that they may be used to wrap the first and second waist regions 12, 14 around wearers having a variety of sizes. As depicted herein the tabs 34, fasteners 36 and target devices 38 have rectangular shapes. As one of ordinary skill in the art will appreciate this is one example of the many shapes that may be used, and the present invention is not intended to be limited to any particular shape and/or size for these parts or to imply that the tabs 34, fasteners 36 and target devices 38 must all have the same general shape and/or size.

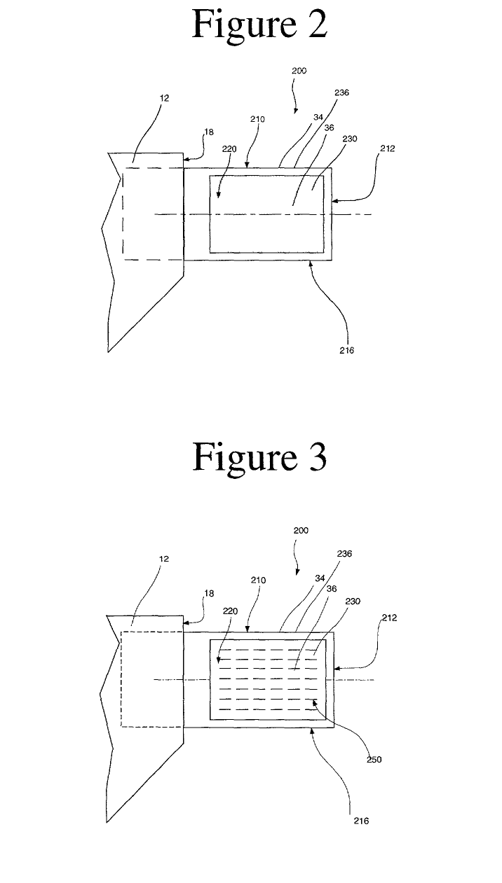

The tabs 34 preferably are attached to the garment 10 at opposite lateral edges 18 of the first waist region 12, and extend distally away from the center of the garment 10. The tabs 34 may be attached to the garment 10 using any method that provides a suitably strong bond. The tabs 34 may be attached in an edge-to-edge fashion with the garment, or the tabs 34 may overlap the top sheet 24 or back sheet 26, or another part of the garment 10, in order to provide a stronger or larger bond area. An overlapping attachment is depicted in FIG. 2, which is a view of a tab with attached fastening element. Preferably, the tabs 34 are attached to one or both of the back sheet 26 and the top sheet 24. Exemplary methods for attaching the tabs 34 to the garment 10 include: ultrasonic welding, thermal bonding, adhesive bonds, stitching, stapling, combinations of the foregoing, and the like. A skilled artisan will be able to apply these methods to attach the tabs 34 to the garment 10 without undue experimentation. Alternatively the tabs 34 may be formed as a part of another component such as the top sheet 24 or back sheet 26 or a combination thereof for example.

The tabs 34 may comprise any suitable material. Nonwoven materials, such as those described elsewhere herein, may be particularly suitable for making the tabs 34. In a preferred embodiment, the tabs 34 are comprised of a material that is easily bonded to the garment 10 and to the fasteners 36 by any of various means known in the art. In some embodiments the tabs 34 may comprise an elastically extensible material. In other embodiments (not shown) each tab may comprise two or more portions made from different materials joined together. For example, each tab 34 may have an elastically extensible portion adjacent lateral edges 18 of waist region 12 and a less elastic portion for the remainder of the tab 34.

Preferably, the fasteners 36 may be sealed and released a number of times without losing their holding and/or fastening ability. The target device 38 may be associated with a surface of the garment 10, or it may be an integral part of the surface of the garment. That is, the surface of the garment may itself act as a target device that will suitably interact with the fasteners 36. The selection and manufacture of fasteners 36, and target devices 38 is known in the art, and a skilled artisan will be able to implement such structures and devices without undue experimentation.

The fasteners 36 may comprise an adhesive tape. In a preferred embodiment, the fastener 36 is a portion of a mechanically interlocking fastener, such as the hook and loop portion of a hook and loop fastener. For hook fasteners, the fastener 36 may comprise a separate strip of material having a backing sheet with the hooks protruding therefrom, or the fastener 36 may protrude directly from the surface of each tab 34. The fasteners 36 also may protrude beyond the laterally distal edges of the underlying tab material. The design and construction of such tabs 34 and fasteners 36 are disclosed, for example, in U.S. Pat. No. 5,624,429 to Long et al., the disclosure of which is incorporated herein by reference in its entirety and in a manner consistent with the present invention.

Also in a preferred embodiment, the target device 38 is the loop portion of a hook and loop fastener that is selected to mechanically engage with the fastener 36 to provide a secure, yet releasable and resealable bond. The target device 38 may be provided as a separate strip of material having a backing sheet with the loops protruding therefrom. In such a case, the target device or devices 38 may be attached to the outermost layer of the garment by any suitable means. In another embodiment, the target device 38 also may comprise a portion of the outer surface material of the garment (whether the outer surface is the back sheet 26 or an outer cover, as discussed elsewhere) that can mechanically engage with the fasteners 36, or that is mechanically or chemically treated to be able to mechanically engage with the fasteners 34.

Because the fasteners 36 or target device 38 may potentially cause irritation of the wearer's skin, in some embodiments such fasteners 36 or target devices 38 may be shielded from contact with the wearer, such as by covering the target 38 or fastener 36 with a protective cover prior to being fastened together. Such a protective cover also may serve to prevent the target devices or fasteners from becoming contaminated and inoperable.

Tab 34 with attached fastener element 36 (collectively the fastener assembly 200), is shown in FIG. 2. The fastener assembly has external fastener assembly sides 210, 212, and 216 (collectively perimeter edge 236). The fastener element 36 may be positioned to be coterminous with the tab 34 at any or all of fastener assembly sides 210, 212, 216 or a portion thereof. Alternatively, the fastener element 36 may be positioned inwardly from any or all sides 210, 212 and 216 with the tab 34 only forming any or all of sides 210, 212 and 218 or a portion thereof. In other embodiments the fastener element may extend beyond the tab 34 edges with the fastener assembly forming a portion of any or all sides 210, 212, 216. The fastener assembly 200 region bounded by perimeter edge 236 is internal portion 230. As shown in FIG. 2 the fastener assembly sides 210, 212 and 212 and 216 are linear, however, fastener assemble sides 210, 212 and 216 may be any of a variety of shapes including curved and stepped e.g. a side having a linear portion, a right angle, a second linear portion and a second right angle followed by a third linear portion; for example.

As shown in FIG. 2 the fastener element 36 is attached to the tab in an attachment region 220. Attachment region as used herein refers to the entire contact surface between the fastener element 36 and tab 34 and any areas between the fastener element 36 and sides 210, 212, and 216. Although the fastener element 36 is secured to the tab in this region, continuous physical attachment over the entire contact surface is not required. In another embodiment (not shown) the fastener element 36 may be formed with a substrate and appended to the tab side 212. In these embodiments the attachment region 220 should be interpreted to be the fastener element with substrate portion of the fastener assembly 200.

As noted above, the materials that typically comprise the preferred hook and loop fastener or commonly used adhesive fasteners often must be relatively rigid materials to provide the desired fastening characteristics. Further, since these fasteners experience significant tensile stress in use, it is necessary to firmly secure the fastener element to the tab. The adhesives typically used for securing the fastener elements often add further stiffness.

The present invention addresses, among other things, the problem of stiffness of the fastener assembly 200 by providing at least one and preferably a plurality of slits in the attachment region 220. The slits provide for curvature of the fastener assemblies 200 in two perpendicular axe. This is in contrast to the tab and fastener assembly 200 shown in FIG. 2 which, when bent, tends to generate a “corrugating” effect that increases its rigidity along an axis perpendicular to the bend. Bending in two axis is characteristic of woven fabrics and is associated with good “hand”, e.g., flexibility properties.

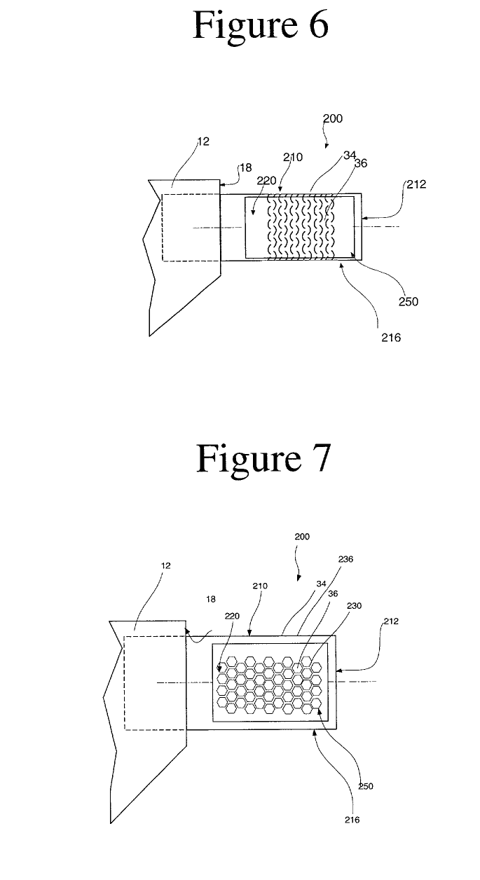

FIGS. 3-7 show exemplary embodiments of fastener assemblies 200 having one or more slits 250. As one of ordinary skill in the art will appreciate, the arrangement of slits shown is exemplary of many arrangements and shapes of slits that may be used in the practice of the invention. Further, for each of the examples shown in FIGS. 3-7 a single type of slit is shown in each example. In other embodiments it may be desirable to use a combination of slit types. These examples are provided as exemplary of the many types and arrangement of slits, and are not intended to suggest that slit types and arrangements are limited to the examples provided.

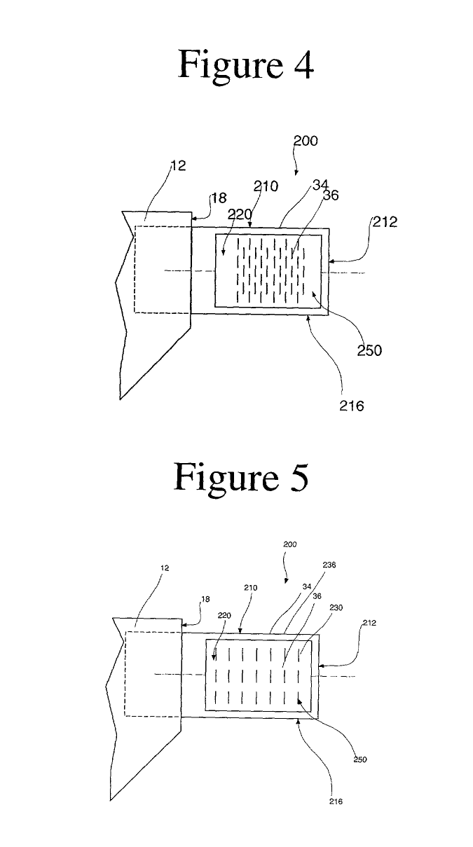

In addition to providing flexibility, the plurality of slits also permits stretching of the fastener assembly 200. Some configurations of slits 250 such as those shown in FIGS. 4, 6 and 7 may permit significant stretching (elasticity). The flexibility and stretching potential of the tab and fastener assemblies 200 having a plurality of slits improves user comfort and facilitates the caregiver securing the fastener including both in positioning the fastener to secure the garment and in physically manipulating the fastener in the process.

The number, shape and orientation of the slits may be varied to obtain desired characteristics of flexibility and stretching potential. In general, increasing the number of slits would be expected to increase the flexibility. On the other hand, too many slits may reduce the structural integrity of the fastener assembly 200. A wide variety of shapes may be used. For example slits may be linear or curved for example. Alternatively, a slit may have more than one linear component such as the hexagonal shape shown in FIG. 7 or more than one curve component or any combination thereof.

Orientation of the slits may also be selected to obtain desired flexibility and stretching potential. Slits arranged in off-setting positions, such as those shown in FIGS. 4, 6 and 7 may have increased flexibility when as compared to columns of slits as shown in FIG. 3, for example. The slits may be oriented such that the slit or portion of the slit aligns with the longitudinal axis 100 of the garment, aligns with the lateral axis 102 of garment or in a position non-parallel to either the longitudinal axis 100 or lateral axis 102, or a combination thereof. In some embodiments it is preferable that the slits be oriented such that the slit or portions of the slit aligns with the longitudinal axis 100 of the garment. For linear arrangements of slits, such as shown in FIGS. 3 and 5 the stretching capacity typically is enhanced in the direction perpendicular to the direction of the slit. Hence in embodiments in which the slit or portion of the slit align with the longitudinal axis 100, stretching capacity is enhanced in the direction parallel to the lateral axis 102. Stretching capacity may be further enhanced if one or more slit ends extend beyond one or more peripheral edge 210, 212, 216 of the fastener assembly 200.

The slits may penetrate the fastener element 36, the tab 34 or both. Typically, greater flexibility and stretching capacity is achieved if the slit penetrates both the tab 34 and fastener element 36. However, in some embodiments, it may be desirable that the slits 250 only penetrate the fastener element 36. For example, in some embodiments the tabs 34 may comprise an elastically extensible material and providing slits in an otherwise relatively rigid fastener element 36 may make the fastener element 36 more compatible with tabs 34.

In other embodiments, slits that penetrate only the fastener element 36 may be desirable to provide more flexibility to the rigid fastener elements while providing the support of a continuous underlying tab material. Slits may be formed in tab or fastener elements supplied to an absorbent garment assembly line, or the slits may be incorporated during the garment assembly process by incorporating a slit cutting step in the process. Conventional cutting equipment may be suitably adapted for this purpose by one skilled in the art.

The invention has been described in connection with the preferred embodiments. These embodiments, however are for example and the invention is not restricted thereto. It will be understood by those skilled in the art that other variations and modifications can easily be made within the scope of the convention as defined in the appended claims.

What is claimed is:

1. An absorbent garment having a longitudinal dimension and a lateral dimension comprising:
a top sheet;
a back sheet, whereby the top sheet and the back sheet form a first waist region, a second waist region longitudinally opposite the first waist region, and a crotch region there between;
an absorbent core disposed between the top sheet and the back sheet;
a pair of tabs, each tab extending laterally away from an opposite lateral side of the first waist region;
at least one fastening element attached to each tab in an attachment region wherein at least one slit is disposed in the attachment region; and
one or more target devices disposed on the garment in the second waist region, where the at least one fastening element and the one or more target devices are capable of attaching to one another, the one or more target devices being located so that the first and second waist regions of the garment may be joined to one another to secure the garment on an intended wearer.

2. The absorbent garment of claim 1, wherein a plurality of slits are disposed in the attachment region.

3. The absorbent garment of claim 2, the region of attachment having a perimeter edge and an internal portion wherein at least one of the plurality of slits is disposed extending from the internal portion through the perimeter edge.

4. The absorbent garment of claim 2, wherein at least a portion of the plurality of slits are disposed in a direction parallel to the longitudinal dimension.

5. The absorbent garment of claim 2, wherein the at least a portion of the plurality of slits are positioned parallel to the lateral dimension.

6. The absorbent garment of claim 2, wherein at least a portion of the slits are disposed in a position non-parallel to both of the longitudinal and lateral dimensions.

7. The absorbent garment of claim 2, wherein at least a portion of the slits are linear.

8. The absorbent garment of claim 2, wherein at least a portion of the slits are curved.

9. The absorbent garment of claim 1, wherein each tab comprises a portion of at least one of the top sheet and back sheet.

10. The absorbent garment of claim 1, wherein the tabs comprise separate pieces of material attached to the garment at or near opposite lateral side edges of the first waist region.

11. The absorbent garment of claim 1, wherein the at least one slit penetrates the fastening element and the tab in the attachment region.

12. The absorbent garment of claim 1, wherein the at least one slit penetrates the fastening element.

13. The absorbent garment of claim 1, wherein the at least one fastening element comprises a hook portion of a hook and loop fastener and the one or more target devices comprise the loop portion of a hook and loop fastener.

14. The absorbent garment of claim 1, wherein the at least one fastening element is an adhesive tape and the one or more target devices comprise a tape receiving surface.

15. A method of making an absorbent garment having a longitudinal dimension and lateral dimension comprising:
providing a top sheet;
providing a back sheet material to overlay the top sheet and thereby form a first waist region, and second waist region longitudinally opposite the first waist region, and a crotch region there between;
providing an absorbent core;
disposing an absorbent core between the top sheet and the back sheet;
providing a pair of tabs, each tab extending laterally away from an opposite lateral side of the first waist region;
attaching at least one fastening element to each tab in an attachment region;
forming at least one slit in the attachment region; and
providing one or more target devices in the second waist region, where the at least one fastening element and the one or more target devices are capable of attaching to one another, the one or more target devices being located so that the first waist and second waist regions of the garment may be joined to one another to secure the garment on an intended wearer.

16. The method of claim 15, wherein each tab comprises a portion of at least one of the top sheet and back sheet.

17. The method of claim 15, wherein providing a pair of tabs comprises attaching separate pieces of material to the garment at or near opposite lateral side edges of the first waist region.

18. The method of claim 15, wherein the at least one fastening element comprises a hook portion of a hook and loop fastener and the one or more target devices comprise the loop position of a hook and loop fastener.

19. The method of claim 15, wherein the at least one fastening element is an adhesive tape and the one or more target devices comprise a tape receiving surface.

20. The method of claim 15, wherein a plurality of slits are formed in the attachment region.

21. The method of claim 20, the region of attachment having a perimeter edge and an internal portion wherein at least one slit of the plurality of slits is disposed extending from the internal portion through the perimeter edge.

22. The method of claim 20, wherein at least a portion of the plurality of slits are formed in a direction parallel to the longitudinal dimension.

23. The method of claim 20, wherein at least a portion of the plurality of slits are formed parallel to the lateral dimension.

24. The method of claim 20, wherein at least a portion of the slits are formed in a position non-parallel to both of the longitudinal and lateral dimensions.

25. The method of claim 20, wherein at least a portion of the slits are linear.

26. The method of claim 20, wherein at least a portion of the slits are curved.

27. The method of claim 15, wherein the at least one slit penetrates the fastening element.

28. The method of claim 15, wherein the at least one slit penetrates the fastening element and the tab attachment region.

29. A fastening system for an absorbent garment, the system comprising:
at least one tab having a plurality of tab sides;
at least one fastening element attached to each tab in a region of attachment wherein at least one slit is disposed in the region of attachment; and
one or more target devices wherein the at least one fastening element and the one or more target devices are capable of attaching to one another.

30. The fastening system of claim 29, wherein a plurality of slits are disposed in the region of attachment.

31. The fastening system of claim 30, the region of attachment having a perimeter edge and an internal portion wherein at least one of the plurality of slits is disposed extending from the internal portion through the perimeter edge.

32. The fastening system of claim 30, wherein at least a portion of the plurality of slits are aligned in a direction parallel to one of the plurality of tab sides.

33. The fastening system of claim 30, wherein a portion of the slits are disposed in a position non-parallel to at least one tab side.

34. The fastening system of claim 29, wherein the at least one fastening element comprises a hook portion of a hook and loop fastener and the one or more target devices comprise the loop portion of hook and loop fastener.

35. The fastening system of claim 29, wherein the at least one fastening element is an adhesive tape and the one or more target device comprise a tape receiving surface.

36. The fastening system of claim 29, wherein the at least one slit penetrates the fastening element.

37. The fastening system of claim 29, wherein the at least one slit penetrates both the fastening element and the tab.

38. A method of making a fastening system, the method comprising:
forming at least one tab having a plurality of tab sides;
attaching at least one fastening element to each tab in an attachment region;
cutting at least one slit in the attachment region; and
providing one or more target devices wherein the at least one fastening element and the one or more target devices are capable of attaching to one another.

39. The method of making a fastening system of claim 38, comprising forming a plurality of slits in the attachment region.

40. The method of making a fastening system of claim 39, the region of attachment having a perimeter edge and an internal portion wherein at least one of the plurality of slits is disposed extending from the internal portion through the perimeter edge.

41. The method of making a fastening system of claim 39, wherein a portion of the plurality of slits are formed in a direction parallel to one of the tab sides.

42. The method of making a fastening system of claim 39, wherein a portion of the slits are formed in a position non-parallel to at least one tab side.

43. The fastening system of claim 39, wherein the at least one fastening element comprises a hook portion of a hook and loop fastener and the one or more target devices comprise the loop portion of a hook and loop fastener.

44. The method of making a fastening system of claim 39, wherein at least a portion of the plurality of slits penetrates the fastening element.

45. The method of making a fastening system of claim 39, wherein the at least a portion of the plurality of slits penetrates both the fastening element and the tab in the attachment region.
